(19) United States
(12) Patent Application Publication
(10) Pub. No.: US 2026/0232243 A1
(43) Pub. Date: Aug. 13, 2026

(54) Extended Hand Training Device

(71) Applicant: Stuart Seymour, Encinitas, CA (US)

(72) Inventor: Stuart Seymour, Encinitas, CA (US)

(21) Appl. No.: 18/936,821

(22) Filed: Nov. 4, 2024

Related U.S. Application Data
(60) Provisional application No. 63/600,357, filed on Nov. 17, 2023.

(51) Int. Cl. A61B 5/22; A61B 5/00; A61B 5/053; A63B 23/16; G06T 17/00

(52) U.S. Cl. CPC A61B 5/225; A61B 5/053; A61B 5/742; A63B 23/16; G06T 17/00; A61B 2562/0219; G06T 2210/41

(57) ABSTRACT

An extended hand training device and corresponding method are used for sensing and measuring a capacitance corresponding to the pressure or force applied by the hand or fingers of the user.

This extended hand training apparatus has protruded slots for the thumb finger, index finger, the middle finger, the ring finger, and the pinky finger. The data obtained from the device can be utilized to create a 3-D model to indicate grip strength or pull force exerted by the hand.

### CROSS-REFERENCE TO RELATED APPLICATION

This application claims benefit of priority to U.S. Provisional Application No. 63/600,357, filed on Nov. 17, 2023, entitled “Extended Hand Training Device,” the entire disclosure of which is incorporated by reference herein.

### FIELD OF DISCLOSURE

The present invention relates generally to capacitive sensing technology in a hand training device, and more particularly to a system, method and computer program utilizing capacitive sensing for determining the force and/or pressure applied to the extended hand training device.

### BACKGROUND

Injuries to the hand and wrist, and recovery from an operation on a patient's wrist, finger, and hand (e.g., carpal tunnel syndrome) often requires implementation of continuous exercise and rehabilitation to increase mobility, reduce corresponding complications, and to reclaim full strength and improved range of motion to the injured region.

Numerous devices are currently offered to support hand/wrist exercises, conditioning, and rehabilitation on the part of the patient and his or her treating therapist. These devices include ring grip exercisers, finger exercisers, hand exercise balls, therapy putty, and hand extension exercisers. Despite the variety of rehabilitation and exercise devices currently available, these devices are not capable of providing results or biofeedback to the therapist and the patient. There is also a need for a device that will keep the patient accountable for completing the exercises as prescribed by his or her physician or therapist.

Other conventional muscular training apparatuses or rehabilitation devices employ magnets or strain gauges to sense and detect the force exerted by a hand on the device. However, sensors that employ magnetic sensing and/or strain gauges are less cost-effective. Additionally, calculating the 3-dimensional shape of an object (i.e., hand) and its movement that exerts a force on a deformable hand training device involves calculations with increased complexity, resulting in higher power consumption that often results in the dead batteries of a battery-operated instrument.

Additionally, there remains a need for a rehabilitation device that is much more convenient and will reduce the number of visits with a doctor. Current rehabilitation devices typically require a patient to visit a therapist anywhere between 5-7 times. Some devices, such as therapy putty, have the potential to lead the patient to performing the wrong exercise.

Accordingly, there is a need for a cost-effective hand rehabilitation apparatus that is capable of keeping the patient accountable for completing prescribed exercises, indicates to the patient whether the exercise is being performed correctly, and consumes much less power when performing these functions.

### SUMMARY

In the case of hand rehabilitations devices, the embodiments of the disclosure solve these problems and overcomes the deficiencies of the prior art. The present invention is an improvement/modification on a previously filed U.S. Non-Provisional application Ser. No. 17/953,294, filed on Sep. 26, 2022, entitled “Hand Training Device.”

The present invention addresses many unmet needs. The present invention generates data for monitoring and can be connected to another device to view the data. The present invention is a training device, a type of biofeedback, that measures real-time grip strength, which can be tracked over time. With this feature, users have measurable strength gains. Furthermore, the present invention captures individual finger and thumb key pinch, push, and pull measurements.

Furthermore, the device is capable of providing an individual digit readout, which a healthcare provider can observe and adjust for proper training and recovery of the patient or user. This differs from other devices that use a dynamometer, which measures the whole hand results on grip strength, instead of individual fingers as done by the present invention.

The present invention serves as medical evaluation: by using the device for the first time you are evaluating the extensor muscles by giving the person/caregiver a digital reading on an app. With each finger the user or medical evaluator will know what the extensor strength is.

The present invention serves for rehab/exercise: following the exercise protocol and after using the device for a period of time the user or healthcare provider can determine if the person/patient is getting better/stronger.

The present invention is a significant advancement over currently available methods to assess patient progress and compliance with rehabilitation goals. The use of the present invention allows remote patient monitoring by any professional provider and adjustment of rehabilitation based on real time data. The present invention represents a substantial leap forward compared to current hand grip dynamometer and key pinch dynamometer.

In an exemplary embodiment of the present disclosure, an extended hand training apparatus comprising: a body comprising a housing and at least one finger resistance device, wherein the finger resistance device protrudes from the housing; at least one capacitive sensor disposed on a surface of the at least one protruded finger resistance device; at least one processor disposed in the housing; and at least one memory including computer program code for one or more programs disposed in the housing, the at least one memory and the computer program code configured to, with the at least one processor, cause the apparatus to: apply a electronic charge to the at least one capacitive sensor; measure a capacitance of the at least one capacitive sensor in real-time, wherein the capacitance is affected by a foreign object; create a 3-dimensional model of the foreign object based on the measured capacitance of the at least one capacitive sensor; generate a deformation model of the at least one protruded finger resistance device based on the 3-dimensional model of the foreign object to obtain a result, wherein the result indicates one of: a grip strength exerted by the hand in at least one axis; a pull strength exerted by the hand in at least one axis, a push strength exerted by the hand in at least one axis, or a pinch strength exerted by the hand in at least one axis; and provide the result as an output.

In one embodiment, the at least one processor is further configured to generate the 3-dimensional shape of the hand based on the number of capacitive sensors and the distance between the capacitive sensors.

A non-transitory computer-readable storage medium carrying one or more sequences of one or more instructions which, when executed by one or more processors, cause an apparatus for generating a deformation model of at least one protruded finger resistance device to perform: retrieving capacitive sensor data, wherein the capacitive sensor data indicates the position of a foreign object or change in position of the foreign object, processing the capacitive sensor data to create a 3-dimensional model of the foreign object, and generating a deformation model of the at least one protruded finger resistance device based on the 3-dimensional model of the foreign object to obtain a result, wherein the result indicates one of: a grip strength exerted by the hand in at least one axis; a pull strength exerted by the hand in at least one axis, a push strength exerted by the hand in at least one axis, or a pinch strength exerted by the hand in at least one axis.

According to another embodiment, a computer-readable storage medium carries one or more sequences of one or more instructions which, when executed by one or more processors, cause, at least in part, an apparatus to detect, in real-time by the at least one capacitive sensor, a position of a hand and a change in the position of a hand in at least one axis; generate a 3-dimensional shape of the hand based on the detected position and detected change of position of the hand; and measure a deformation of the at least one protruded finger resistance device to obtain a result, wherein the result indicates one of: a finger's muscle strength exerted by the hand in at least one axis; or a pinch strength exerted by the hand in at least one axis.

In addition, for various example embodiments of the invention, the following is applicable: a method comprising facilitating a processing of and/or processing (1) data and/or (2) information and/or (3) at least one signal, the (1) data and/or (2) information and/or (3) at least one signal based, at least in part, on (or derived at least in part from) any one or any combination of methods (or processes) disclosed in this application as relevant to any embodiment of the invention.

In certain embodiments, the protruded finger placement is retractable.

In certain embodiments, the protruded finger placement is not retractable.

In certain embodiments, the protruded finger placement can be adjusted to user's finger length.

In certain embodiments, the protruded finger placement can be ring-shaped or loop-shaped as observed in FIG. 5.

In certain embodiments, the protruded finger placement can be fingertip shaped, as observed in FIG. 6.

In certain embodiments, one or more straps can be used to secure and/or stabilize the device to hand and/or arm.

In certain embodiments, the device has Bluetooth connectivity and connects to a computer, mobile device, or table via Bluetooth to send data.

In certain embodiments, the device has a one or more accelerometers and/or one or more gyroscope to record motion from the wrist, elbow, and/or shoulder.

In certain embodiments, a brace is used to secure the device to the hand and/or arm.

For various example embodiments, the following is applicable: a method comprising the means for determining the force applied comprising the steps performed by the processor of the apparatus of any of the claims.

These and other systems, methods, objects, features, and advantages of the present invention will be apparent to those skilled in the art from the following detailed description of the preferred embodiment and the drawings. All documents mentioned herein are hereby incorporated in their entirety by reference.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The embodiments of the invention are illustrated by way of example, and not by way of limitation, in the figures of the accompanying drawings:

FIG. 1 illustrates a perspective view of the extended hand training apparatus configured to measure the finger's muscle strength, grip strength, and/or pinch strength exerted by a hand, in accordance with an example embodiment.

FIG. 2 illustrates a perspective view of the extended hand training apparatus, in accordance with an example embodiment, with the protruded finger placement retracted.

FIG. 3 illustrates a perspective view of the extended hand training apparatus, in accordance with an example embodiment, with the protruded finger placement retracted.

FIG. 4 illustrates a perspective view of the extended hand training apparatus, in accordance with an example embodiment, with the protruded finger placement retracted.

FIG. 5 illustrates a perspective view of the extended hand training apparatus with configured to measure the finger's muscle strength, grip strength and/or pinch strength exerted by a hand, in accordance with an example embodiment.

FIG. 6 illustrates a perspective view of the extended hand training apparatus configured to measure the finger's muscle strength, grip strength and/or pinch strength exerted by a hand, in accordance with an example embodiment.

### DETAILED DESCRIPTION

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments. As used herein, the terms “data,” “content,” “information,” and similar terms may be used interchangeably to refer to data capable of being displayed, transmitted, received and/or stored in accordance with embodiments of the present invention. Thus, use of any such terms should not be taken to limit the spirit and scope of embodiments of the present invention.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect. Reference will now be made in detail to the preferred embodiments of the invention.

The current invention disclosure is an extended hand training apparatus comprising: a body comprising a housing and at least one protruded finger resistance device connected to the housing; at least one capacitive sensor disposed on a surface of the at least one protruded finger resistance device; at least one processor disposed in the housing; and at least one memory including computer program code for one or more programs disposed in the housing, the at least one memory and the computer program code configured to, with the at least one processor, cause the apparatus to: detect, in real-time by the at least one capacitive sensor, a position of a hand and a change in the position of a hand in at least one axis; generate a 3-dimensional shape of the hand based on the detected position and detected change of position of the hand; and measure a deformation of the at least one protruded finger resistance device to obtain a result, wherein the result indicates one of: a grip strength exerted by the hand in at least one axis; a pull strength exerted by the hand in at least one axis, a push strength exerted by the hand in at least one axis, or a pinch strength exerted by the hand in at least one axis.

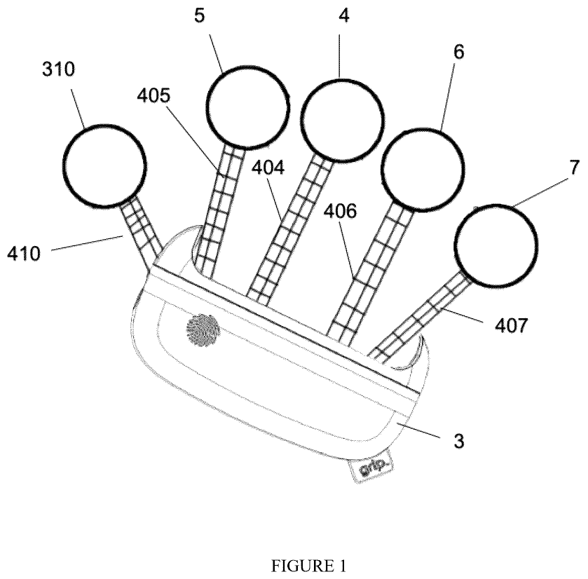

FIG. 1 illustrates a perspective view of various components of the extended hand training apparatus for analyzing data relative to the capacitive proximity sensor units, according to one embodiment.

In one embodiment, the extended hand training device may include a sensor system (e.g., an array of capacitive proximity sensors disposed on the surface of multiple protruded finger resistance device connected to the housing, etc.), the interface device, and a remote device. For purposes of illustration, rather than limitation, the extended hand training apparatus may be described as measuring the grip, pull, push, and pinch strength of a user's hand when the user grips the extended hand training apparatus and applies a force to each individual protruded finger resistance device. In a preferred embodiment, the extended hand training apparatus number is comprised of five (5) protruded finger resistance devices, a finger resistance device for the thumb 310, index 5, middle 4, ring 6, and pinky 7. It may be appreciated that the extended hand training apparatus may be used for other measurements. FIG. 1 also illustrates, fully extended telescopically thumb finger protrusion 410, index finger protrusion 405, middle finger protrusion 404, ring finger protrusion 406, and pink finger protrusion 407. It may be appreciated that the extended hand training apparatus may be used for other measurements.

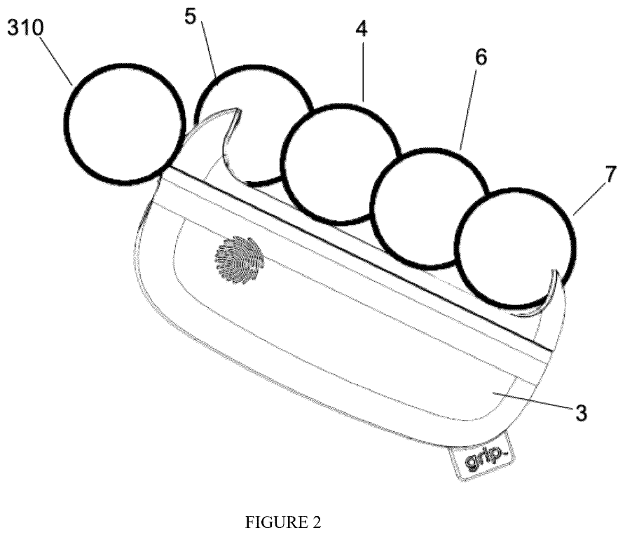

FIG. 2 illustrates boot 3 installed on the device with the protrusions fully retracted. The user can grab the device and rest his or her fingers on the appropriate slots.

In another embodiment, the sensor system may include at least one capacitive proximity sensor unit and a biofeedback device. The capacitive proximity sensor unit may include an array of conductive materials separated by one of more dielectric materials. The individual conductive materials are connected to a microcontroller, capable of being rapidly charged and sense the change in capacitance between any subset of such conductive materials. The capacitance is affected by charge-carrying materials, such as a human hand, which allows the ability to sense the proximity of a conductive material such as a hand. By rapidly alternating the charge and capacitive measurement cycle among the subsets of conductive materials connected to the microcontroller, the change in the proximity of other charge-carrying materials can be calculated.

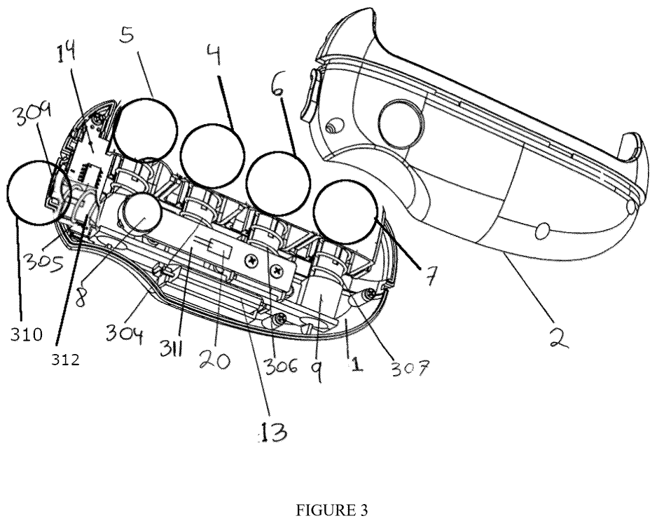

FIG. 3 shows case-A 2 disconnected from case-A 1. FIG. 3 illustrates the internals in a preferred embodiment of the device. Connection port 309 serves for charging the device, as well as for transferring data. The device has PCBA-main 14 (printed circuit board), compression spring 305, compression spring 304, compression spring 306, and compression spring 307. The device measures the force exerted by the user on plunger-mid-L 4, plunger-mid-R 6, plunger-side-L 5, and plunger-side-R 7. Thumb plunger 310. Support plate 311 holds button (pinch) 8; button (pinch) 8 is used to detect key pinch measurements. Support plate 311 also holds strain gauge 350 Ohm 20. Guide plunger 9 holds compression springs 305, 304, 306, and 307. Guide plunger 9 also serves as a guide for plunger-mid-L 4, plunger-mid-R 6, plunger-side-L 5, plunger-side-R 7, and thumb plunger 310.

The interface device may include a capacitance measurement module, a calculation module, a voltage control module, the microcontroller, memory, or a biofeedback module. The microcontroller may include the voltage control module. The memory may be, but not limited to, a single memory, ROM, RAM, EEPROM, optical storage, or any other non-volatile or non-transitory storage medium capable of storing digital data. The capacitance measurement module, calculation module, and voltage control module could reside in RAM memory, flash memory, registers, or any other form of writable computer-readable storage medium known in the art including non-transitory computer-readable storage medium. The remote device may include a display and user input, and may include the processors and computing devices of, for example, a smart phone or personal computer, as known in the art. In other embodiments, the microcontroller may include both analog and digital circuitry to perform the functionality of the capacitance measurement module, the calculation module, the voltage control module, and the biofeedback module. In some embodiments, the interface device may comprise a processing device, such as a microprocessor or central processing unit, a controller, special-purpose processor, digital signal processor, or one or more other processing devices known by those of ordinary skill in the art.

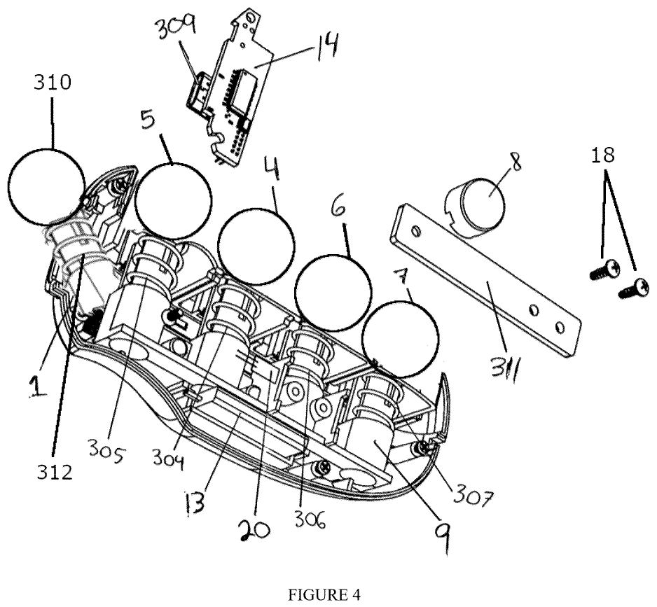

FIG. 4 illustrates a partial exploded view of the device showing case-A 1, connection port 309, PCBA-main 14 (printed circuit board), plunger-mid-L 4, plunger-mid-R 6, plunger-side-L 5, plunger-side-R 7, and thumb plunger 310 not pictured. Guide plunger 9 holding compression springs 305, 304, 306, 307 and 312, which come into contact with corresponding plunger-mid-L 4, plunger-mid-R 6, plunger-side-L 5, plunger-side-R 7, and thumb plunger 310. Support plate 311 is showed exploded from the device; button (pinch) 8 connects to support plate 311 and plate 311 is attached using screws 18 into guide plunger 9.

The processor may include one or more processing cores with each core configured to perform independently. A multi-core processor enables multiprocessing within a single physical package. Examples of a multi-core processor include two, four, eight, or greater numbers of processing cores. The processor and accompanying components have connectivity to the memory. The memory includes both dynamic memory (e.g., RAM, magnetic disk, writable optical disk, etc.) and static memory (e.g., ROM, CD-ROM, etc.) for storing executable instructions that when executed perform the inventive steps described herein to provide mobility insight data related to shared vehicles for a POI. The memory also stores the data associated with or generated by the execution of the inventive steps.

In use, for example, a user grips the extended hand training apparatus, and each finger applies a force to each individual protruded finger resistance device. In the case of an embodiment with retractable protruded finger resistance devices, the force can be applied in extended mode or retractable mode. Each finger resistance device has at least one capacitive proximity sensor connected to the microcontroller. By aligning the capacitive proximity sensors in 3-D space, the extended hand training apparatus is capable of determining the position of a hand, the change in position of the hand, or the change in distance between the capacitive proximity sensors. Such construction, for example, enables determining the amount and distribution of deformation of the extended hand training apparatus when the user's hand applies pressure or force to the individual finger resistant devices.

Measuring the deformation of the extended hand training apparatus enables the apparatus to measure the grip, pull, push, and pinch strength applied by the user's hand. To record the grip, pull, push, and pinch strength, distinct subsets of an array of conductive materials of the capacitive proximity sensor are successively selected and rapidly charged by the voltage control module. The capacitance measurement module measures the capacitance of a capacitive element, such as a copper pad. In an embodiment, the capacitance measurement module and the voltage control module can be disposed in the housing of the extended hand training apparatus and coupled to the capacitive proximity sensor unit via wires. In another embodiment, the capacitance measurement module can measure the capacitance(s) or differential capacitance in terms of voltage or current. In another embodiment, the capacitance measurement module then transmits voltage data or current data to the calculation module.

In an embodiment, the calculation circuit analyzes the values of the voltage data or current data provided by the capacitance measurement module to calculate, for example, directional information of a foreign object, such as the position of a hand and the relative distance of the capacitive elements of the capacitive proximity sensor units. The placement of these capacitive elements determines the set of 3-dimensional directions. The calculation module is capable of performing measurements. The number of directions and therefore the resolution of the 3-dimensional position of the foreign object, i.e., a human hand, is determined by the number of capacitive sensors and their distance. The 3-dimensional resolution can then be aligned to measure the deformation of an arbitrarily shaped object, such as the extended hand training device. The grip, pull, push, and pinch strength are then measured indirectly based on the measured deformation of the extended hand training apparatus. The calculation module may then transmit the measured grip and pinch strength and directional data to the memory (which then becomes logged data) and the control and analysis software installed on a user device. The user device may include a display and/or a user input, such as input keys.

In some embodiments, parameters such as maximum limits (and minimum limits) may be input through, for example, the user device and transferred from the user device to the biofeedback module via, for example, wireless technology (e.g., over a wireless local area network (WLAN) such as a Bluetooth network or Wi-Fi network) or transferred via mini-USB ports or the like, as known to one of ordinary skill in the art. In another embodiment, a physician, doctor, surgeon, or therapist may input the max/min parameters onto a cloud-based service provider, which are automatically downloaded by the user device and then transmitted to the biofeedback module. As such, if the user does meet the desired parameters or undesired, as the case may be, the biofeedback module may transmit the logged data to the control and analysis software and then uploaded to the cloud-based service provider, for the purposes of review by the physician, doctor, surgeon, or therapist.

In certain embodiments, the biofeedback device may produce a notification to the user that a predefined input parameter has been reached, such as the maximum or minimum deformation or maximum or minimum force the user's hand may apply to the extended hand training apparatus, so that the user understands in real-time the limits relative to the movement of the user's hand, for example. The notification may be at least one of a visual notification, an audible notification, a tactile notification, or some other notification to facilitate the user's understanding of the user's maximum or minimum limits prescribed by a doctor, for example. Alternatively, the notification can be any combination of visual, audible, and tactile notifications. The visual notification may be in the form of a blinking (or various colored) light, or a light displayed on the sensor system itself or the extended hand training apparatus and/or also may be visualized on a display of the user device. The audible notification may be a ring or beep or the like that may preferably be audibly transmitted from the extended hand training apparatus but may also be transmitted from the sensor system. The tactile notification may be coupled to or integrated with the sensor system or disposed in the shell of the extended hand training apparatus. Such tactile notification may be in the form of a vibration or some other tactile notification. In this manner, the biofeedback device may notify the user in real time to ensure proper form is applied and that the extended hand training apparatus is used properly by the patient in accordance with prescribed orders by a doctor, physician, or therapist. Similarly, in another embodiment, a user may input parameters of a minimum/maximum force into the user device for biofeedback notification. Further, in another embodiment, the user may input parameters for both a minimum deformation and a maximum angular deformation. Alerting the user or patient in real-time as to whether the patient is performing the hand rehabilitation with the device correctly and in accordance with the doctor's prescription may prevent re-injury to the patient's hand and, thus, reduce the number of visits with a doctor

In another embodiment, upon completing a session of rehabilitation therapy or training or the like, for example, logged data may be stored in the memory or storage device of the housing of the extended hand training apparatus. The memory stores various data including force applied data, hand position data, grip strength data, pull strength, push strength, and pinch strength data. The logged data may then be transferred to a remote device. The remote device may be any known computing device, such as a mobile device, smart phone, tablet, personal computer, gaming system, etc. In one embodiment, the logged data may be transferred to a smart phone via, for example, wireless technology (e.g., over a wireless local area network (WLAN) such as a Bluetooth network or Wi-Fi network) or transferred via mini-USB ports or the like, as known to one of ordinary skill in the art. In another embodiment, the logged data may be transferred to a personal computer via a port, such as a USB port with, for example, a portable memory device, such as a thumb drive. The user may then save the logged data on the remote device for further analysis. In another embodiment, the logged data may be transmitted to a cloud-based service provider, for the purposes of storing the logged data in the cloud in batches or in real-time and to be retrieved by a physician, doctor, surgeon, therapist, or insurer at a later time for review and analysis of the patient's rehabilitation progress. The user may also save several sessions of logged data to the remote device to obtain further analysis and comparison data to better understand, for example, progress or regress in the rehabilitation of the user's hand. Allowing the user or patient and doctors access to the log may be useful for increasing adherence to prescribed exercises and accountability tracking to ensure the patient is successfully rehabilitated.

The extended hand training device and the electronics disposed inside the housing may be powered by numerous power sources that include one or more of batteries, rechargeable batteries, wired power, and capacitive storage, among others.

The term non-transitory computer-readable medium is used herein to refer to any medium that participates in providing information to the processor, including instructions for execution. Such a medium may take many forms, including, but not limited to, non-volatile media, volatile media, and transmission media. Non-volatile or non-transitory media include, for example, optical or magnetic disks, such as storage device. Volatile media include, for example, dynamic memory. Transmission media include, for example, coaxial cables, copper wire, fiber optic cables, and carrier waves that travel through space without wires or cables, such as acoustic waves and electromagnetic waves, including radio, optical and infrared waves. Signals include man-made transient variations in amplitude, frequency, phase, polarization, or other physical properties transmitted through the transmission media. Common forms of computer-readable media include, for example, a floppy disk, a flexible disk, hard disk, magnetic tape, any other magnetic medium, a CD-ROM, CDRW, DVD, any other optical medium, punch cards, paper tape, optical mark sheets, any other physical medium with patterns of holes or other optically recognizable indicia, a RAM, a PROM, an EPROM, a FLASH-EPROM, any other memory chip or cartridge, a carrier wave, or any other medium from which a computer can read.

In one embodiment, a non-transitory computer-readable storage medium carrying one or more sequences of one or more instructions (e.g., computer code) which, when executed by one or more processors (e.g., the processor as described in FIG. 4), cause the extended hand training apparatus to perform any steps of the various embodiments of the methods described herein.

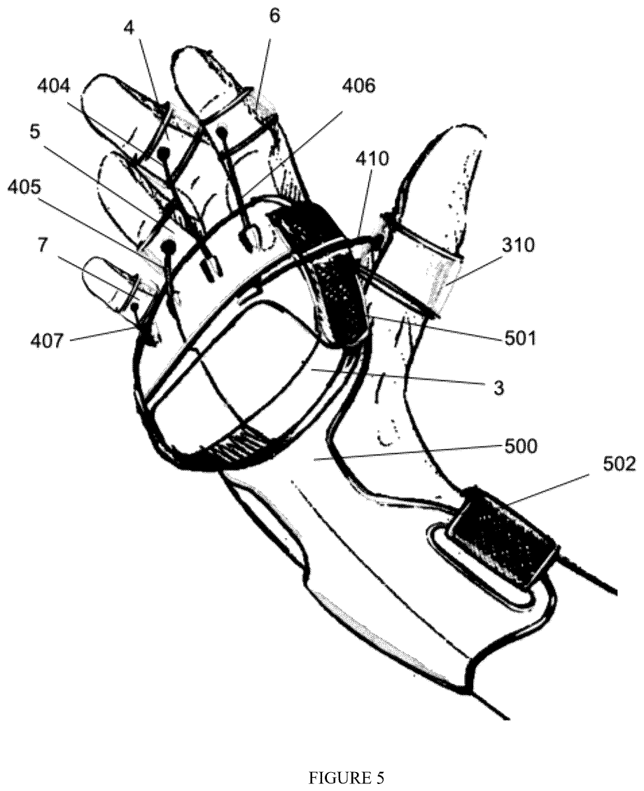

FIG. 5 illustrates a perspective view of various components of the extended hand training apparatus for analyzing data relative to the capacitive proximity sensor units, according to one embodiment.

In one embodiment, the extended hand training apparatus is comprised of five (5) protruded or extended ring-shaped finger resistance devices, a ring-shaped finger resistance device for the thumb finger 310, index finger 5, middle finger 4, ring finger 6, and pinky finger 7. FIG. 5 also illustrates, almost fully extended (in an elastic embodiment of the device) thumb finger protrusion 410, index finger protrusion 405, middle finger protrusion 404, ring finger protrusion 406, and pink finger protrusion 407.

It can also be observed that brace 500, arm strap 502 and device strap 501. Brace 500 serves to stabilize the device, which is inside the boot 3. The boot 3 serves as a cover for the device. Arm strap 502 and 501 serve to hold the device and brace 500 in a preferred position.

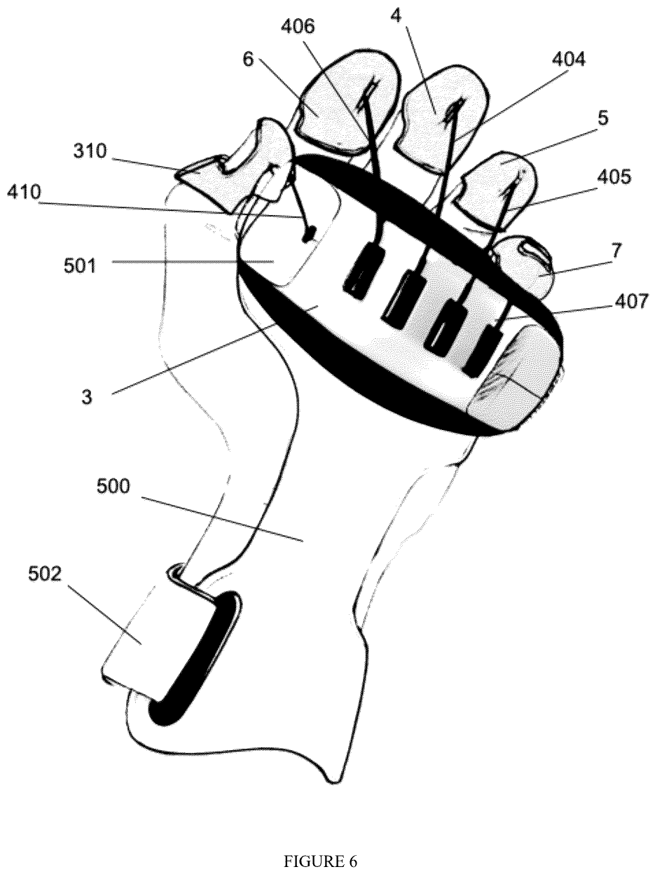

FIG. 6 illustrates a perspective view of various components of the extended hand training apparatus for analyzing data relative to the capacitive proximity sensor units, according to one embodiment.

In one embodiment, the extended hand training apparatus is comprised of five (5) protruded or extended fingertip-shaped finger resistance devices, a fingertip-shaped finger resistance device for the thumb finger 310, index finger 5, middle finger 4, ring finger 6, and pinky finger 7. FIG. 6 also illustrates, not fully extended (in elastic embodiment of the device) thumb finger protrusion 410, index finger protrusion 405, middle finger protrusion 404, ring finger protrusion 406, and pink finger protrusion 407.

It can also be observed that brace 500, arm strap 502 and device strap 501. Brace 500 serves to stabilize the device, which is inside the boot 3. The boot 3 serves as a cover for the device. Arm strap 502 and 501 serve to hold the device and brace 500 in a preferred position.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

What is claimed is:

1. A hand training device comprising:
a body,
a housing,
at least one protruded finger resistance device,
at least one capacitive sensor,
at least one processor, and
at least one memory configured to:
store at least one computer program code configured to:
with the at least one processor to cause the hand training device to apply an at least one electronic charge to the at least one capacitive sensor, and
measure a capacitance of the at least one capacitance sensor.

2. The at least one protruded finger resistance device from claim 1, wherein the at least one protruded finger resistance device is adjustable and fully retractable.

3. The at least one computer program from claim 2, wherein at least a first program is configured to:
create a 3-dimensional model of a foreign object based on the measured capacitance of the at least one capacitive sensor, when the at least one capacitance is affected by the foreign object and
generate a deformation model of the at least one finger resistance device based on the 3-dimensional model of the foreign object.

4. The at least a first program from claim 3, wherein it provides a one or more result indicating a grip strength exerted by the foreign object in the at least a one axis and provides the one or more result as an output.

5. The at least a first program from claim 3, wherein the at least a first program provides a one or more result indicating a pinch strength exerted by the foreign object in the at least a one axis and provides the one or more result as an output.

6. The at least first program from claim 3, wherein the at least one processor is configured generate a 3-dimensional model of a foreign object based on the measured capacitance of a two or more capacitive sensor and the distance between the two or more capacitance sensors, when the at least two or more capacitance sensors are affected by the foreign object.

7. The at least first program from claim 3, wherein the at least first program is configured to receive data from a one or more accelerometer.

8. The at least first program from claim 3, wherein the at least first program is configured to receive data from a one or more gyroscope.

9. A method for acquiring haptic data from a one or more protruded finger resistance device and displaying results comprising:
a non-transitory computer-readable storge medium,
a one or more sequences of a one or more instruction,
a one or more processor,
a non-transitory computer-readable storage medium carrying the one or more sequence of the one or more instruction which, when executed by the one or more processors, causes an outcome.

10. The method from claim 9, wherein the outcome consists of causing an apparatus for generating a deformation model of at least one protruded finger resistance device to perform:
retrieving a one or more capacitive sensor data, wherein the one or more capacitive sensor data indicates the position of a foreign object or a change in position of the foreign object, processing the one or more capacitive sensor data to create a 3-dimensional model of the foreign object, and
generating a deformation model of the at least one protruded finger resistance device based on the 3-dimensional model of the foreign object to obtain a result,

11. The method of claim 10, wherein the result indicates a grip strength exerted by the hand in a at least one axis.

12. The method of claim 10, wherein the result indicates a pinch strength exerted by the hand in the at least one axis.

13. The method of claim 9, wherein the outcome consists of causing an apparatus to detect, in real-time by the at least one capacitive sensor,
a position of a hand and a change in the position of the hand in at least one axis;
generate a 3-dimensional shape of the hand based on the detected position, and detected change of position of the hand; and
measure a deformation of the at least one protruded finger resistance device to obtain a result.

14. The method of claim 13, wherein the result indicates a grip strength exerted by the hand in at least one axis.

15. The method of claim 13, wherein the result indicates a pinch strength exerted by the hand in at least one axis.

16. The method of claim 9, wherein the haptic data is acquired from a one or more accelerometer.

17. The method of claim 9, where in the haptic data is acquired from a one or more gyroscope.
